(19) United States
(12) Patent Application Publication
(10) Pub. No.: US 2019/0060206 A1
(43) Pub. Date: Feb. 28, 2019

(54) ENHANCEMENT OF ALGINATE FILM INTEGRITY THROUGH USE OF MICA

(71) Applicant: L'OREAL, Paris (FR)

(72) Inventors: David CHAN, Edison, NJ (US); Jonathan James FAIG, Sayreville, NJ (US)

(73) Assignee: L'OREAL, Paris (FR)

(21) Appl. No.: 15/692,039

(22) Filed: Aug. 31, 2017

(51) Int. Cl. A61K 8/73; A61K 8/25; A61K 8/02

(52) U.S. Cl. CPC A61K 8/733; A61K 8/0212; A61K 8/25

(57) ABSTRACT

A method of improving film integrity of a mask having alginate comprises (a) applying a mask composition onto a surface, said mask composition comprising alginate, one or more micas, and one or more solvents; and (b) exposing the mask composition to a crosslinking solution comprising polyvalent cations of one or more metals for a time sufficient to crosslink the alginate, whereby the mask is formed. A kit for making a mask comprises a) the mask composition and b) the crosslinking solution. An alternative kit comprises a) a solution comprising alginate; b) one or more micas; and c) the crosslinking solution.

### FIELD OF THE INVENTION

The invention relates to mask compositions and uses thereof.

### BACKGROUND OF THE INVENTION

Masks such as facial masks can be of any of a variety of kinds for different purposes: deep-cleansing, by penetrating the pores; healing acne scars or hyper-pigmentation; or brightening, for a gradual illumination of the skin tone. Some masks are designed to dry or solidify on the face, while others remain wet. The desired effects of a facial mask treatment may include revitalizing, healing, or refreshing, and the benefits may be temporary or long-term.

Duration for wearing a mask varies with the type of mask and the manufacturer's usage instructions, and may range from a few minutes to overnight. After use, the masks are removed by rinsing off with water, wiping off with a damp cloth, or peeling off. While it is particularly advantageous to be able to simply peel the mask off, it has been difficult to do so with known mask compositions in one piece without leaving pieces or residue behind. Therefore, improvements in the physical strength of masks would be a welcome advance in the cosmetic art.

### SUMMARY OF THE INVENTION

The invention provides a method of improving film integrity of a mask having alginate, comprising:
(a) applying a mask composition onto a surface, said mask composition comprising alginate, one or more micas, and one or more solvents; and
(b) exposing the mask composition to a crosslinking solution comprising polyvalent cations of one or more metals for a time sufficient to crosslink the alginate, whereby the mask is formed.

The invention also provides a mask made by this method. The thickness of the mask may be 1-10 mm, and the mask may have a tensile force at break that is at least 10% higher than that of an analogous mask not comprising any mica.

Crosslinking may occur within one minute. The crosslinking solution may be applied to the mask composition by spraying.

The mask composition may be a cream, paste or gel.

The polyvalent cations in the crosslinking solution may comprise calcium ions.

The mask composition may be free of added calcium ions.

The surface may be a skin surface, wherein the mask has a shape conformed to the skin surface.

The mask composition may not comprise any clay.

The one or more solvents may comprise water, and may further comprise glycerin.

The invention also provides a kit for making a mask, comprising
a) a mask composition comprising alginate and one or more micas; and
b) a crosslinking solution comprising polyvalent cations of one or more metals;

wherein the crosslinking solution is capable of crosslinking the alginate.

The invention also provides a kit for making a mask, comprising
a) a solution comprising alginate;
b) one or more micas; and
c) a crosslinking solution comprising polyvalent cations of one or more metals;

wherein the crosslinking solution is capable of crosslinking the alginate after the one or more micas are incorporated into the solution.

The polyvalent cations in the crosslinking solution of either of the above kits may comprise calcium ions.

### DETAILED DESCRIPTION OF THE INVENTION

The inventors have found that crosslinked films containing alginate and one or more micas and prepared as described herein have improved film integrity, as indicated by, for example, force at break during tensile strength testing with a texture analyzer. The films are suitable for use as masks, for example, cosmetic or therapeutic masks.

A two-step method is provided to improve film integrity of a mask having alginate. The method comprises 1) applying a mask composition comprising alginate, one or more micas and one or more solvents to a surface, and then 2) applying a crosslinking solution to the mask composition under conditions, for example, at ambient temperature and humidity, for a time sufficient to crosslink (or cure) the alginate. As a result, a mask with improved film integrity is formed.

The mask compositions may be applied to any surface. The surface may be a user's face or to other skin surfaces, including for example, the lips or areas on the chin, neck, hands, arms, or legs, which can be prone to drying, especially under low humidity conditions. The mask compositions may be applied to the skin without being embedded in, adhered to, or otherwise supported by a woven or nonwoven mesh, fabric, or other support.

The mask composition may be in any physical form that allows easy application of the mask composition to a surface. The mask composition may have a shape conformed to the surface (e.g., skin surface). For example, the mask composition may be a cream, paste or gel. The mask composition may be a layer on the surface, having a thickness of no more than 10 mm, or no more than 9, 8, 7, 6, 5, or 4 mm. The thickness may be at least 1 mm, or at least 2 or 3 mm. The thickness may be 1-10 mm, for example, 3 mm.

Upon crosslinking, the mask composition on the surface is cured and forms a mask. The thickness of the mask may be no more than 10 mm, or no more than 9, 8, 7, 6, 5, or 4 mm. The thickness may be at least 1 mm, or at least 2 or 3 mm. The thickness may be 1-10 mm, for example, 3 mm. The mask may have a shape conformed to the surface (e.g., a skin surface). The inclusion of mica in the crosslinked mask composition provides an enhancement effect, such that tensile force at break of the mask may be at least 10% higher than that of an analogous mask not containing any mica, or at least 15%, 20%, 25%, 30%, or 35% higher. The term “analogous mask not containing any mica” means a mask made from an otherwise identical mask composition, but for the absence of mica, applied and contacted with the same crosslinking solution under the same conditions. There is no particular upper limit to the strength increase, which may be at most 100%. As used herein, discussion of the tensile force at break for a mask having a shape conformed to a skin surface of a user may be understood to mean the tensile force at break of a free-standing, flat version of the same mask.

The mask compositions and crosslinking solutions are described below in terms of the types and amounts of essential and optional ingredients used. Unless otherwise specified or made apparent by the context, all percentages herein are on a weight basis. If a percentage is identified as being on a dry basis, the basis is the total of all of the ingredients other than water.

### Mask Composition

The mask composition includes alginate, one or more micas, and one or more solvents. Auxiliary ingredients may also be included. The mask composition may have little or no polyvalent metal cation content. For example, the content may be insufficient to crosslink the alginate. The maximum content of polyvalent metal cations in the mask composition may be 1.0%, 0.5%, 0.2%, 0.1%, 0.05%, 0.02%, or 0.01% on a weight basis, relative to the amount of alginate. These numbers all refer to polyvalent metal cations, together or individually, other than those that may be naturally present in the one or more micas and/or the alginate. The mask composition may be free of added polyvalent metal cations (e.g., calcium ions), which means the mask composition does not comprise any polyvalent metal cations (e.g., calcium ions) other than those naturally present in the alginate and/or the one or more micas.

Alginate

Alginates of any variety are suitable for use. While sodium alginate may be used, potassium or ammonium alginate may be used instead or in addition. The alginate may constitute at least 0.5% of the composition, or at least 1, 1.2, 1.5, or 1.7%. It may constitute at most 15%, or at most 7, 6, 5, 4, 3, 2.5, or 2.3%. When alginate is referred to herein as an ingredient in a composition, it refers to uncrosslinked alginate unless otherwise stated or made clear from the context. Crosslinking of the alginate may be detected visually by increased striations in the gel network and turbidity, as well as via a sensorial tightening effect.

Micas

Exemplary suitable micas include di-micas (e.g., illite and muscovite) and tri-micas (e.g., biotite), and one or more micas may be the only minerals present in the mask composition or the mask. The micas may have an average particle size of at least 0.2 μm, or at least 0.5, 1, 2, 5, or 10 μm. The particle size may be at most 60 μm, or at most 50, 40, 30, or 20 μm. One specific mica suitable for use is an aluminium fluoro magnesium sodium silicate (CAS 12001-26-2) mica product having a particle size of 9.48 μm, sold under the tradename SUBMICA E by Sensient Cosmetic Tehnnologies. The one or more micas may, in total, constitute at least 0.5% of the mask composition, or at least 1, 1.5, 2, or 2.5%. They may constitute at most 15%, or at most 10, 9, 8, 7, 6, 5, 4, or 3.5%.

Solvents

The mask composition includes one or more solvents, which may include water and/or organic solvents, for example, water-soluble or water-miscible solvents. Specific exemplary organic solvents include ethanol, glycerin, 1,3-propanediol, propylene glycol, butylene glycol, pentylene glycol, and caprylyl glycol. If organic solvents are included, they may, in total, constitute at least 1% of the composition, or at least 2, 3, 4, 5, 6, 7, or 8%. They may constitute at most 40%, or at most 25, 15, or 10%.

Water may constitute at least 10% of the composition, or at least 15, 20 or 25%. It may constitute at most 90%, or at most 80, 70, 60, or 50%.

Auxiliary Ingredients

The mask composition may include or exclude any of a variety of auxiliary ingredients or additives to achieve a desirable effect, for example, a desirable cosmetic or therapeutic effect, or ease of use. Nonlimiting examples of auxiliary ingredients include colorants, odorants, vitamins (e.g., tocopherol), chelating agents, various active agents, aesthetic modifiers, and preservatives. Nonlimiting examples of the preservatives include chlorphenesin, phenoxyethanol, and caprylyl glycol. Other examples of auxiliary ingredients include alpha-hydroxy acids, carboxylic acids, halocarboxylic acids, dicarboxylic acids, limonene-based oil, hyaluronic acid, ammonium hyaluronic acid, gelatin, starch, cellulose gums, xanthan gum, guar gum, polyvinyl alcohols, maltodextrin, carrageenan, agar, chitosan, poly (N-vinyl lactam), acrylate polymers, methacrylate polymers, oat flour, beta-1,3 glucan-type polysaccharides, pollen corpuscles, vinyl pyrrolidone (co)polymers, and other water-soluble polymers. Solum diatomeae (diatomaceous earth), silica, and other particulate inorganic materials are also examples of auxiliary ingredients.

Further examples of auxiliary ingredients include aesthetic modifiers. Examples include aluminum starch octenylsuccinate, derivatized celluloses (e.g., hydroxypropylmethylcellulose, hydroxyethylcellulose), xanthan gum, sclerotium gum, locust bean gum, pectin, acrylates/C₁₀-C₃₀ alkyl acrylate crosspolymers, ammonium acryloyldimethyltaurate homopolymers, ammonium acryloyldimethyltaurate/vinylpyrrolidone copolymers, nylon 12 particles, and synthetic waxes.

If present, the auxiliary ingredients may in total constitute at least 0.5% of the mask composition, or at least 1, 1.5, 2, or 2.5%. They may constitute at most 10%, or at most 9, 8, 7, or 6%.

The mask composition can be prepared by simply combining the various ingredients.

### Crosslinking Solution

The crosslinking solution is a solution, typically aqueous, containing polyvalent cations of one or more metals, for example, trivalent or divalent metal cations. Examples include Ca, Cu, Zn, Al, and Fe (ferrous or ferric) cations, as well as Zr-containing cations. For example, any of these may be present as a salt. The salt may be one with an inorganic counterion, for example, nitrate, chloride, sulfate, or phosphate. Or, the counterion may be an organic one, for example acetate, ascorbate, citrate, or pidolate.

The salts or other compounds containing the polyvalent metal cations may constitute at least 0.1 wt % of the crosslinking solution, or at least 0.5, 1, 2, or 5 wt %. They may constitute at most 30 wt % of the solution, or at most 25, 20, 15, or 10 wt %.

To achieve a desirable effect, for example, a desirable cosmetic or therapeutic effect or ease of use, the crosslinking solution may also include or exclude any of the solvents and/or any of the auxiliary ingredients described above for use in the mask composition.

### Applying the Mask and Crosslinking Solutions

The mask compositions according to the invention may have a creamy texture that is easy for the user to apply evenly by hand. Once applied to a surface, the mask composition may be converted to a mask by exposing the mask composition to the crosslinking solution, for example by spraying on the crosslinking solution, using any spray device known in the cosmetic art. The crosslinking solution can be applied immediately after the mask composition is applied onto the surface, or it can be applied at a later time if desired. Crosslinking occurs rapidly, for example, within about five minutes or even one minute or less, and the resulting mask can be left in place for as long or as short a time as desired. The mask may be kept in place for at least 5 minutes, or at least 10, 15, 20, or 30 minutes. There is no particular maximum time for leaving the mask in place, for example, it may be at most 10 hours. At the end of the desired time, the mask may be easily removed by peeling, leaving very little residue on the surface (e.g., a skin surface). Typically, the mask can be peeled from a user's skin in at most three pieces, or at most two pieces. More typically, it can be peeled in a single piece.

Kits are provided for making the mask of the present invention. A kit may include
a) a mask composition comprising alginate and one or more micas; and
b) a crosslinking solution comprising polyvalent cations of one or more metals;
wherein the crosslinking solution is capable of crosslinking the alginate.

Alternatively, a kit may include
a) a solution comprising alginate;
b) one or more micas; and
c) a crosslinking solution comprising polyvalent cations of one or more metals;
wherein the crosslinking solution is capable of crosslinking the alginate after the one or more micas are incorporated into the solution.

### Examples

Comparative Example A analogous mask composition and Example B mask composition were prepared by blending together the ingredients shown in Table 1. The ALGIN was sodium alginate sold under the tradename ALGINATE DE SODIUM (POLY MANNURONATE ET GULURONATE) (Manufacturer/Distributor: FMC Corporation; Distributed by SOLIANCE (GIVAUDAN)). MICA was an aluminium fluoro magnesium sodium silicate (CAS 12001-26-2) mica product having a particle size of 9.48 μm, sold under the tradename SUBMICA E by Sensient Cosmetic Technologies.

TABLE 1
Mask Compositions

| | Comp. Example A | Example B |
|---|---|---|
| MICA | | 3 |
| GLYCERIN | 9.3 | 9.3 |
| WATER | 88.7 | 85.7 |
| ALGIN | 2 | 2 |

Each of the mask compositions was spread on a glass plate to a thickness of 200 μm using a drawdown rod, and then immediately sprayed with a crosslinking solution of 5% aqueous calcium chloride at a rate of 5 grams solution per 100 cm² of mask composition coating. The resulting films were allowed to crosslink for five minutes at ambient temperature and humidity, and then peeled away from the glass plate for strength testing.

Film strength was evaluated in triplicate for Comparative Example A and Example B using a texture analyzer in tensile testing mode, using a 5-cm wide strip of film.

TABLE 2
Film Strength

Force at Break (g)

| | Comp. Example A | Example B |
|---|---|---|
| 1 | 119.0 | 184.0 |
| 2 | 125.0 | 149.0 |
| 3 | 117.0 | 161.5 |
| Avg. | 120.3 | 164.8 |
| Std. Dev. | 4.16 | 17.7 |

As can be seen in Table 2, the average force at break was 37% higher for Example B than for Comparative Example A, providing an enhancement effect on strength or film integrity. The cured mask of inventive Example B was also noticeably more ductile than that of Comparative Example A.

Comparative Examples C and D were prepared and tested as in Example B, except that the mica was replaced with an equal amount of laponite clay and kaolin clay, respectively. The film strength testing results for three samples of each of Examples C and D are shown in Table 3.

TABLE 3
Film Strength

Force at Break (g)

| | Comp. Example C | Comp. Example D |
|---|---|---|
| 1 | 105.5 | 93.0 |
| 2 | 116.0 | 91.0 |
| 3 | 90.0 | 91.5 |
| Avg. | 103.8 | 91.8 |
| Std. Dev. | 13.1 | 1.0 |

As seen in Table 3, mask compositions using laponite and kaolin, both of which are clays, failed to produce the enhancement of strength provided by mica.

Comparative Example E and Examples F and G mask compositions were prepared as shown in Table 4.

TABLE 4

| | Comp. Example E | Example F | Example G |
|---|---|---|---|
| ACTIVE COMPOUNDS | 0.2 | 0.2 | 0.2 |
| MICA | — | 1 | 5 |
| ALGIN | 2 | 2 | 2 |
| POLYMER | 0.1 | 0.1 | 0.1 |
| PRESERVATIVES | 0.7 | 0.7 | 0.7 |
| SOLVENTS | 13 | 13 | 13 |
| WATER | 84 | 83 | 79 |

The mask compositions were tested as in Example B. The film strength testing results for three samples of each are shown in Table 5, measured as force at break in grams on a 5-cm wide strip of film.

TABLE 5

| | 1 | 2 | 3 | Avg. | Std. Dev. |
|---|---|---|---|---|---|
| Comp. Example E | 42 | 57.5 | 31.5 | 43.66667 | 13.07988 |
| Example F | 97 | 79.5 | 114 | 96.83333 | 17.2506 |
| Example G | 84 | 108.5 | 77.5 | 90 | 16.34778 |

As seen in Table 5, including mica at even a 1% level resulted in a dramatic increase in tensile strength or film integrity compared with an analogous composition without mica.

Although the invention is illustrated and described herein with reference to specific embodiments, the invention is not intended to be limited to the details shown. Rather, various modifications may be made in the details within the scope and range of equivalents of the claims without departing from the invention.

What is claimed:

1. A method of improving film integrity of a mask having alginate, comprising:
(a) applying a mask composition onto a surface, said mask composition comprising alginate, one or more micas, and one or more solvents; and
(b) exposing the mask composition to a crosslinking solution comprising polyvalent cations of one or more metals for a time sufficient to crosslink the alginate, whereby the mask is formed.

2. The method according to claim 1, wherein the thickness of the mask is 1-10 mm, and wherein the mask has a tensile force at break that is at least 10% higher than that of an analogous mask not comprising any mica.

3. The method according to claim 1, wherein the crosslinking occurs within one minute.

4. The method according to claim 1, wherein the crosslinking solution is applied to the mask composition by spraying.

5. The method according to claim 1, wherein the mask composition is a cream, paste or gel.

6. The method according to claim 1, wherein the polyvalent cations comprise calcium ions.

7. The method according to claim 1, wherein the mask composition is free of added calcium ions.

8. The method according to claim 1, wherein the surface is a skin surface, and wherein the mask has a shape conformed to the skin surface.

9. The method according to claim 1, wherein the mask composition does not comprise any clay.

10. The method according to claim 1, wherein the one or more solvents comprise water.

11. The method according to claim 10, wherein the one or more solvents further comprise glycerin.

12. A kit for making a mask, comprising
a) a mask composition comprising alginate and one or more micas; and
b) a crosslinking solution comprising polyvalent cations of one or more metals;
wherein the crosslinking solution is capable of crosslinking the alginate.

13. The kit according to claim 12, wherein the polyvalent cations comprise calcium ions.

14. A kit for making a mask, comprising
a) a solution comprising alginate;
b) one or more micas; and
c) a crosslinking solution comprising polyvalent cations of one or more metals;
wherein the crosslinking solution is capable of crosslinking the alginate after the one or more micas are incorporated into the solution.

15. The kit according to claim 14, wherein the polyvalent cations comprise calcium ions.

16. A mask formed by the method according to claim 1.

17. A mask formed by the method according to claim 2.

18. The mask according to claim 16, wherein the mask can be peeled from a user's skin in at most three pieces.

19. The mask according to claim 17, wherein the mask can be peeled from a user's skin in at most three pieces.
